(19) United States
(12) Patent Application Publication
(10) Pub. No.: US 2011/0276125 A1
(43) Pub. Date: Nov. 10, 2011

(54) Medical Devices, Methods of Producing Medical Devices, and Projection Photolithography Apparatus for Producing Medical Devices

(76) Inventors: Jeffrey P. WALKER, Clovis, CA (US); Lev Drubetsky, Vancouver (CA)

(21) Appl. No.: 13/099,908

(22) Filed: May 3, 2011

Related U.S. Application Data
(60) Provisional application No. 61/331,803, filed on May 5, 2010.
(60) Provisional application No. 61/348,110, filed on May 25, 2010.
(60) Provisional application No. 61/348,210, filed on May 25, 2010.

(51) Int. Cl. A61F 2/82; G03B 27/52; G03F 7/20

(52) U.S. Cl. 623/1.15; 430/320; 355/55

(57) ABSTRACT

Stents and other medical devices that can have specific geometric configurations (curves, contours, tapers) and/or patterns (e.g., grooves) thereon, methods of making such medical devices, and apparatuses for making such medical devices are disclosed. Projection photolithography is used to define patterns the medical devices. The methods can form grooves, ridges, channels, holes, wells, and other geometric patterns (e.g. parallelograms such as squares, rectangles and other trapezoids; triangles, pentagons, spirals, hexagons, etc.) on the surface of both the inner and outer diameters (e.g., on both the inner and outer surfaces) of stents or other cylindrical, tubular or curved-surface medical devices, allowing the manufacture of stents having customized geometry/contours on all surfaces, which can minimize endothelial surface disruption of blood flow.

### RELATED APPLICATIONS

This application claims the benefit of U.S. Provisional Patent Appl. Nos. 61/331,803, filed May 5, 2010 (Attorney Docket No. JPW-001-PR); 61/348,110, filed May 25, 2010 (Attorney Docket No. SON-002-PR); and 61/348,210, filed May 25, 2010 (Attorney Docket No. SON-003-PR), each of which is incorporated herein by reference in its entirety.

### FIELD OF THE INVENTION

The present invention relates to stents and other medical devices that can have specific patterns and geometric configurations (curves, contours, tapers) on the inner and/or outer surfaces thereof, methods of manufacturing such stents and medical devices, and an apparatus capable of forming such stents and medical devices with a high level of precision.

### DISCUSSION OF THE BACKGROUND

In vascular medicine, the use of stents in blood vessels has been highly successful in reducing both short-term and long-term complications of balloon angioplasty (e.g., elastic recoil, restenosis) and several generations of stents have evolved along a continuum of bare-metal stents, drug-eluting stents, and future biodegradable stents. Although the rates of re-intervention procedures are generally low, complications such as thrombosis, delayed healing, and/or non-incorporation of stent struts into the vascular wall continue to result in significant morbidity and mortality, requiring local drug delivery and/or prolonged systemic anti-platelet therapy to achieve acceptable therapeutic outcomes. These technologies are highly expensive and have measurable adverse side effects that contribute to patient morbidity and mortality.

The causes of stent thrombosis and delayed vascular healing are multi-factorial but are due, in part, to the disruption of local hemodynamic flow around the implanted stent structure. Recent work has described that the current generation of stent struts protrude a significant portion of their strut profile (round, square or rectangular) into the blood flow. There are significant adverse effects of blood flow disturbance caused by the shape and/or configuration of the implanted stent that, in turn, causes disruption of normal endothelial cell function. Analysis of blood flow patterns in and around embedded stent struts has demonstrated that there are significant areas of flow disruption that can lead to reduced shear stress on endothelial cells. Since shear stress is a product of flow velocity and plasma and blood viscosity, when flow velocity is reduced, blood flow separation (e.g., disruption of laminar blood flow) occurs in both the proximal and distal portions of the inserted stent strut. Reduced shear stress is correlated with reduced production of potent vasodilators such as nitric oxide and/or prostacyclin, etc., along with the up-regulation of a number of mitogenic compounds including NF-KB. It is likely that the separation areas create recirculation zones that are prone to platelet aggregation and activation along with the expression of cellular mitogens, both of which will also contribute to the induction of smooth muscle cell proliferation and restenosis. Thus, there is a need to reduce the stent strut profile and the disruption of normal blood flow caused by stent strut protrusion.

Additionally, there is a need to improve the two-dimensional (planar) geometry of the stent in order to improve stent flexibility as well as the magnitude and distribution of radial forces on the artery. Stents must possess a number of critical design features in order to be commercially viable. First, they must possess intrinsic radial strength following deployment, enabling the stent to resist normal compression forces that tend to close the stented tube/cylinder. These forces can be intrinsic forces, mediated by smooth muscle cells in the vessel wall, or extrinsic forces that attempt to close the stented vessel by severe flexion, torsion or other movements.

Stents must also balance the need for high radial strength with the need for flexibility, which allows stents to transition through tight radii and obstructions in the vasculature, respiratory, urinary or GI organ systems. Success in driving innovative flexibility designs, while maintaining radial strength, is highly advantageous because flexibility is an important determinant in trackability and ease of delivery. Traditionally, coil stent designs such as the Wiktor stent have been the most flexible stents. Among the slotted tube designs, the MultiLink designs have had the lowest stiffness (see, e.g., Ormiston, J. A., et al., Stent longitudinal flexibility: A comparison of 13 stent designs before and after balloon expansion. Catheterization and Cardiovascular Interventions, 2000. 50(1): p. 120-124). It is understood that overall flexibility of delivery systems is a function of balloon, catheter and stent stiffness, and in most systems, balloon stiffness is greater than stent stiffness. However, stent flexibility is critically important following stent deployment and is thus a major factor in commercial and clinical success.

A further requirement for effective stents is that they must be conformable to their final surrounding location, able to fit into curved structures with minimal malapposition, or gap between the stent and the wall of the stented structure. Conformability is a separate design feature from flexibility but related in that the stent must be free to expand in the radial direction without collapse. Failure of the stent to fully expand and fill a curved structure can lead to adverse events in the stented structure that could include thrombosis, migration, and/or inflammation.

A number of approaches have been used to address the issues discussed above. These approaches have provided some improvement to stent designs. However, there are still problems that need to be addressed. For instance, to prevent disruption hemodynamic flow, designers have employed advanced alloys to reduce stent strut thickness. The contributions of strut thickness, metal, artery ratio, and stent cell configuration, and their relationship to intimal hyperplasia and restenosis have been well articulated in the literature and have lead to some improvements in stent designs that more evenly distribute force loads on the vessel wall. The long-term biocompatibility of metals embedded in arterial walls has been established for a variety of metals including stainless steel and cobalt-chromium alloys. However, despite these improvements, delayed healing and acute thrombosis continue to present major problems for patients and clinicians and require inflexible and aggressive anti-platelet drug therapies for weeks to months following stent implantation.

Current commercial manufacturing methods for stents are not able to solve the difficulty of constructing hemodynamically designed stent struts with contoured surfaces, particularly on the internal diameter (ID) of the stent strut. Current processes use lasers (YAG, CO₂) to cut stent shapes from tubular/cylindrical substrates and are only able to be applied to the outer diameter (OD) of the cylinder. An alternative methodology involves welding together a series of sinusoidal rings to create the stent. However, these processes are incapable of controlling the geometric contour of stent struts on both inner and outer diameters with micron-scale precision in the absence of photolithographic methodologies.

Recent patent applications and issued patents have recognized the importance of streamlined strut architecture. For example, Berglund et al. (U.S. Patent Application Publication No. 2008/0306581) discusses a contoured wing-like stent strut that minimizes turbulent flow, similar to the leading wing of an aircraft. However, in the disclosure of Berglund, there is little discussion about how these struts might be manufactured. There is a general reference to mechanical abrasion (e.g., sand blasting), but the implementation of, e.g., a high-volume manufacturing process capable of process control to achieve a desired shape and/or contour of a stent at the micron scale is not disclosed.

In another example, Pacetti (U.S. Pat. No. 6,685,737) references a similar curved surface for both an endoluminal and an abluminal stent strut contour in order to minimize pressure on the endothelial cell surface while minimizing flow disturbances on the luminal surface. Pacetti discusses the use of post-process techniques to smooth away an initial starting contour that is a complex polygonal shape using an adaptation of laser cutting technology in which either the laser or the stent is rotated along both longitudinal and radial axes to square off the stent edges. However, lasers do not cut edges; rather they melt material and controlling with micron-scale precision a melting process would be highly difficult and impractical.

The post-process techniques described in the '737 patent to smooth away contour from a relatively complex polygonal shape may require significant and time-consuming modification and be relatively impractical both in terms of controllability and time required to achieve efficient product output. In fact, none of the published patent applications or patents appear to describe a methodology for producing stent strut contours and stent architecture that effectively minimizes blood flow disruption (see, e.g., FIG. 8 of the '737 patent). Therefore, a new methodology providing stents that reduce blood flow disruption, have improved flexibility and conformability, and that are capable of being manufactured efficiently is needed.

Previously described photolithographic techniques proposed for stent manufacture have utilized shadow techniques, wherein the mask is placed either in direct contact with or in proximity to a stent. The resolution of such systems is around 3-5 μm under optimum circumstances, and the systems are very sensitive to particulate contamination and mechanical damage.

For example, the application of photolithography to cylindrical surfaces has been described in Hines (U.S. Pat. No. 6,274,294). Hines discloses an apparatus for exposing a pattern onto a photoresist-coated cylinder, using a precision contact photolithography that is directly applied onto the photoresist-coated surface of the cylinder. However, the apparatus of Hines is only suitable for application of a pattern to the outer diameter (OD, or outer surface) of the tubular substrate and uses contact photolithography to transfer the process from the photomask to the cylindrical substrate. The method disclosed by Hines for stent manufacturing is shadow printing. In shadow printing, a mask and stent either are in direct contact with one another (contact printing) or in close proximity (proximity printing). The resist is then exposed by a nearly collimated beam of UV through the back of the mask. The intimate contact between mask and resist can provide a resolution of approximately 1 μm. However, the technique requires a flexible mask for stent application (e.g., as described in U.S. Pat. No. 6,274,294). Additionally, contact printing suffers a major drawback in that dust particles, and other debris can be transferred from the mask to the stent surface. Particulate matter such as dust particles can become embedded in or adhere to the mask and cause permanent damage to the mask. This results in image defects on an exposed resist, and subsequent defects in the mask pattern and stents produced thereafter with each succeeding exposure. To minimize mask damage, the proximity exposure method is the most practical of the two methods. The proximity printing method is similar to the contact printing method, except that there is a small gap 10-50 μm between the stent and the mask during exposure. However, the small gap results in optical diffraction at the edges of the photo mask, making control of small features difficult, particularly at the micron scale due to diffraction-induced degradation of the optical resolution. This methodology would be difficult and impractical for high-throughput manufacturing, and does not permit modification of the inner strut contours.

Karol (U.S. Pat. No. 3,645,179) describes a method for exposing a photoresist on the inside and outside of a cylinder using shadow photolithography. This technique requires placing photoresist on the inner and outer surfaces of a cylinder, and then placing a mask in close proximity to each surface. Exposure of the photoresist is accomplished by placing the cylinder into a device that passes a light source up through and around the cylinder, exposing the photoresist through the mask using shadow photolithography. However, there are practical limits to using this manufacturing technology with smaller diameter tubes such as coronary stents (stents with diameters of ˜2-3 mm). The methodology is further complicated by mask fragility and particulates, as described above. It would be difficult to translate this process into a high-volume manufacturing operation and impossible to apply art (e.g., a pattern) to the inner surface of a stent having such a small diameter.

A recent publication authored by de Miranda et al. (“Fabrication of TiNi thin film stents,” Smart Mater. Struct. 18 (2009) 104010) describes a method for adapting photolithographic methods to apply patterns on cylindrical substrates. In the system disclosed by de Mirada, shadow photolithography is used to pass light across a moving photomask, under which a cylinder is spun along its long axis, transferring the photomask image to the photoresist-coated cylinder. This process is capable of micron-scale resolution, but is impractical for scaled-up manufacturing. Furthermore, this process is incapable of applying a pattern to the inner diameter of the stent.

Alternate forms of applying photoresist patterns on the OD of tubular substrates such as laser photolithography and electron beam lithography could also be used to apply a surface pattern and/or geometry to a cylindrical object, but would be unable to apply a pattern and/or geometry to the inner surfaces of the cylinder. Additionally, all of these forms of lithography involve the use of masks in very close proximity to the imaged substrates and thus are complicated by particulate contamination of the masks and/or fragility of the masks due to frequent manipulation. Consequently, these methodologies are highly complicated, expensive, and result in relatively low-throughput manufacturing processes.

None of the above-mentioned manufacturing methods is capable of addressing a modification of the stent strut geometry, particularly in terms of modification of the internal (luminal) surface of a stent strut. Producing a hemodynamic shape on the luminal surface that differs from the shape on an outer diameter of a stent is not possible using conventional techniques of stent manufacture. Thus, there is a need for a robust, high-throughput, micron-scale precision manufacturing method that can produce stents with contoured strut geometries on both the inner and outer strut surfaces.

Despite the present limitations on strut manufacturing, the concept of adding grooves to the stent surface has been described in several patents and patent applications relating to drug delivery, radio-opacity of stents and for fluid drainage. U.S. Pat. No. 4,307,723 describes the creation of at least one external longitudinal groove to provide a passage for fluid from the distal end of a ureteral stent to the proximal end. U.S. Patent Application Publication No. 2009/0248137 A1 describes the use of holes or grooves, known as “wells,” that open onto the exterior surface and are considered suitable for containing one or more therapeutic agents. The wells are described as variable in depth, opening onto the OD of the stent, or passing through to the interior (ID) of the stent and containing therapeutic material. U.S. Pat. No. 6,471,721 describes forming at least one groove along a tube, inserting radiopaque material into the groove, securing the material to the groove and then cutting the tube into a particular pattern to form the struts of a stent. However, none of these patents/applications have mentioned or considered the possibility of using grooved structures to alter mechanical deformability of the stent or stent struts, enhancing flexibility along an axial dimension.

### SUMMARY OF THE INVENTION

The present invention is directed to stents and other medical devices that can have specific geometric configurations (curves, contours, tapers) thereon, methods of making such stents and other medical devices, and apparatuses for making such stents and other medical devices. Applications include, but are not limited to, vascular stents used in treating diseases including arterial and venous patency in atherosclerotic vascular disease, pulmonary arterial stenoses, coarctation, and pulmonary and systemic venous obstruction, among other applications.

The present invention includes the application of projection photolithography to produce customized grooves, geometry, and contours on any or all surfaces of stent struts, achieving a manufacturing solution for producing stents designed specifically to minimize endothelial surface disruption of blood flow, and have improved flexibility and conformability. The invention permits the creation of geometric patterns or surface topographies such as grooves, ridges, channels, holes, wells, and other geometric patterns (e.g. parallelograms such as squares, rectangles and other trapezoids; triangles, pentagons, spirals, hexagons, etc.) on the surface of both the inner and outer diameters (e.g., on both the inner and outer surfaces) of stents or other cylindrical, tubular or curved-surface medical devices.

The disclosed projection photolithography techniques allow for the incorporation of more hemodynamic and aerodynamic features into stent design will result in a significant diminution or elimination of blood flow disruptions around stent struts and other medical devices, facilitating earlier healing and incorporation of stents into the vascular wall. Also, improved stent flexibility and conformability, balanced with high radial strength, will improve healing, reduce the demand for anti-platelet therapies, and avoid acute thrombosis, thereby enabling solutions for significant problems experienced by patients that have stent implantations.

The present invention also presents a design and method for reducing stent strut diameter (thickness), in addition to reducing strut dimensions, by incorporating two or more nested stents (e.g., stent segments and/or layers) in a concentric superstructure to form a scaffolded stent. The stent layers may be joined by one or more connection points that are formed using a bonding process, such as diffusion bonding, welding, etc., and the stent segments in a given layer may be joined by one or more strut-like connectors (see, e.g., FIG. 6). Unlike most other commercial designs that utilize a single scaffold comprised of many design elements, the present invention describes multiple, ultra-thin stent layers that are assembled into a larger superstructure that is implanted as a whole unit. The multilayer, nested structure reduces strut thickness in each layer without necessarily sacrificing radial strength of the stent by increasing numbers of layers in the stent. As a result, the radial stiffness of each layer of the stent decreases, thereby decreasing the bending stiffness significantly (i.e., the stent is very flexible), but the cumulative stiffness of the stent is equivalent to stiffness of conventional single-layer or solid stents. In addition, the radial force on the stent can be distributed through many contact points (thereby reducing contact stress).

Using projection lithography to project light onto the inner and outer stent surfaces enables the formation of a stent from a single tube or cylinder with a customized radius, curvature, contours, surface properties, length, and width on each individual stent strut on both the inner diameter (ID) and outer diameter (OD) of the stent. The ability to apply predetermined patterns to the internal surfaces of the struts is a major advantage over conventional techniques. Alternatively, the present projection lithography apparatus and method can be used on a flat sheet, foil or film of a medically- and/or biologically-acceptable material (e.g., a biologically-acceptable metal or alloy), which can be rolled (or curved) and welded after etching. The present methods allow the patterning of intricate 3D shapes on both the external and internal strut surfaces that can be achieved by a series of exposures and etchings (e.g., to a controlled depth). The utility of such patterning may include, but is not limited to, promotion or inhibition of cellular migration, cellular adhesion, cell shape, cell-based sensing, patterns of tissue growth, cellular differentiation, cellular apoptosis, and cellular chemistry. Stents typically have a mesh design, with a great deal of open area within the structure. Applying patterns to the internal surface of the struts is possible, even where the metal fill factor (i.e., Σ_{area of the struts}/τ_{area of the openings}) is relatively low. Metal coverage area of stents generally ranges from 8% to 24%, but a majority of stents are between 11-18% when fully expanded, resulting in the ability to pass light around stent struts opposite the face of the inner surface to be patterned. FIG. 2 illustrates the small amount of shadowing that occurs as a result of a stent strut 21 placed in line with a projection lithography system designed to etch contours on the inner surface 22 of a given strut.

Another advantage of the system is that the photomask is located some distance away from the target, resulting in an extended mask life since there is little to touch or damage the mask. The mask (see, e.g., mask 4 in FIG. 1) is hidden inside of the system at some distance from the stent surface and can be manufactured at a larger scale (4:1 scale, for example). This allows for a less expensive mask manufacturing process. Additionally, the mask is may be scaled significantly larger than the dimensions of the pattern to be produced, making the mask easier to manufacture as well as minimizing the effects of mask errors, contamination by particulates and mask motion errors. This design allows for an extremely high resolution can be achieved which can be calculated by the formula CD=kᵢ·λ/NA where CD is the minimum feature size that can be resolved by the system, k₁ is a process related coefficient determined by the photoresist used (as is known in the art), λ=wavelength of light (300 nm) and NA is a numerical aperture of the imaging lens. For NA=0.2, the smallest feature/resolution that can be imaged is 1.5 μm. The depth of focus calculation is given by DOF=k₂·λ/NA² where k₂ is a process related constant known in the art (e.g., 0.5). In the presently disclosed system, the depth of focus would be approximately ±3.75 μm. In order to maintain the depth of focus, the distance between the imaging lens and the tube surface may be controlled in the present apparatus through the use of an auto-focus system, an example of which would be a laser range finder and ultra-precision stage (see, e.g., FIG. 1).

Projection lithography will enable the application of any desired geometry and/or pattern to an individual stent strut using a traditional material etching or removal method as is known in the art. Additionally, material may be added to the stent struts and nodes by using additional coating steps in a multi-step coating-imaging-etching process. This allows the projection lithography methodology to serve as a microfabrication technique to add contours and/or materials to stent strut surfaces, giving it additional flexibility for manufacturing novel, biocompatible shapes. The photolithography system described herein can also be used in combination with microcontact printing techniques such as soft lithography to create patterns on the surfaces of medical devices. For example, FIG. 3 graphically depicts a profile of a stent strut that is etched from a material having a rough or non-curved surface (e.g., from a rectangular substrate) to a strut having an arc shaped or curved surface through series of steps involving coating, pattern transfer, exposing and etching (and optionally, pattern removal and/or repeating using a different pattern).

The stents described herein may be used for a number of applications, including arterial and venous patency in atherosclerotic vascular disease, pulmonary arterial stenoses, coarctation, pulmonary and systemic venous obstruction, among other applications. Significant improvements in stent flexibility, conformability and clinical performance can be driven at the “macro” scale by using strut configuration, strut thickness, choice of metals, and stent architecture (multiple, nested strut cell designs; closed vs. open; coil vs. slotted vs. ring; etc). However, significant enhancements in stent performance can be gained by the incorporation of “micro” scale features (e.g., grooves and patterns in the ID and OD of the struts) that change both the physical characteristics of the stent as well as the biological interface of the stent with the body. Additionally, the incorporation of hemodynamic and aerodynamic flow principles (e.g., curved edges) into strut design can result in significant enhancement of stent performance in terms of reducing flow disruptions that contribute directly to thrombosis, inflammation, and restenosis.

Additionally, deep grooves and/or channels can be used to create discrete points of increased flexibility and conformability along the length of a stent, inscribed on both the inner diameter (ID) and outer diameter (OD) of the stent using projection photolithography. Both the ID and OD of a stent can be patterned using projection photolithography to create surface features down to the micron scale (e.g., with resolution down to a single micron). By etching at discrete strut intersections (nodes) on both ID and OD, a “bellows-type” flexible joint can be created to allow increased flexibility and motion along the strut length, while maintaining radial strength (see, e.g., FIG. 5). Also, stent conformability can be significantly enhanced by the use of multiple etched grooves or other strut modifications in the OD of the stent at points along the stent struts to allow maximum deformability of the strut during stent expansion, thus enabling struts to fit to vessel contours.

Embodiments of the invention provide a method of forming a patterned material on a medical device, comprising coating at least part of the medical device with a photoresist, transferring a pattern to the photoresist using projection photolithography, and developing the photoresist with a developer, thereby forming the patterned material on the medical device.

Embodiments of the invention also provide a method of forming a patterned material on an inner surface of a photoresist coated medical device, comprising coating the inner surface of the medical device with a photoresist, passing radiation through a mask, thereby exposing one or more portion of the photoresist to the radiation, and developing the photoresist with a developer, thereby forming the patterned material on the inner surface of the medical device.

Embodiments of the invention also provide a method of forming a medical device, comprising coating at least part of the medical device with a photoresist, patterning the photoresist using projection photolithography to form grooves or other features or shapes on both the inner diameter and the outer diameter of the medical device, and developing the photoresist with a developer, thereby forming a patterned surface on the inner diameter and the outer diameter of the medical device.

Embodiments of the invention also provide a method of creating three-dimensional surfaces on an inner surface and an outer surface of a medical device or part thereof, comprising forming a patterned photoresist on each of an inner surface and an outer surface of the medical device or part thereof, and selectively removing a material of the medical device or part thereof exposed by the patterned photoresist, or selectively adding a new material to a surface of the medical device or part thereof exposed by the patterned photoresist.

Embodiments of the invention also provide a method for creating three-dimensional surfaces on a medical device or part thereof, comprising forming a patterned photoresist on the surface of the medical device or part thereof using projection photolithography, and selectively removing a material of the medical device or part thereof exposed by the patterned photoresist, or selectively adding a new material to a surface of the medical device or part thereof exposed by the patterned photoresist.

Embodiments of the invention also provide an apparatus for making a medical device, comprising a radiation source providing a radiation beam, a range finder configured to enable locating a surface of the medical device, a focusing lens for focusing the radiation beam onto the medical device, and a first mechanical stage configured to move the medical device rotationally and/or along at least one of two orthogonal axes, a first one of the orthogonal axes being parallel with an optical axis of the apparatus, the first mechanical stage having sufficient precision to enable focusing the radiation beam on either inner surface of the medical device under a first set of imaging conditions and on an outer surface of the medical device under a second set of imaging conditions.

Another object of the invention is to provide a medical device, comprising one or more cylindrical bodies having a mesh-like wall including struts and strut nodes, and a first pattern of surface features on inner surfaces of the struts and/or strut nodes. The medical device may optionally include a second pattern of surface features on outer surfaces of the struts and/or strut nodes.

Further embodiments of the invention provide a tubular medical device, comprising a thin metal wall having outer diameter of in a range of about 0.5 mm to 50 mm and a metal wall thickness in range of about 10-150 μm (e.g., 50-125 μm, 75-100 μm, or any value or range of values therein), struts in the metal wall having lengths in a range of about 0.1-3 mm (e.g., 1 mm, or any value or range of values therein), gaps in the metal wall between the struts, and grooves on an inner surface and an outer surface of the tubular medical device.

Further embodiments of the invention provide a medical device, comprising first and second cylindrical bodies, each having a mesh-like wall including struts and strut nodes, wherein the cylindrical bodies are bonded together in a concentric nested arrangement. Optionally, a first pattern of surface features may be on inner surfaces of the struts and/or strut nodes in one or more of the cylindrical bodies. The medical device may optionally include a second pattern of surface features on outer surfaces of the struts and/or strut nodes of one or more of the cylindrical bodies.

Another object of the invention is to provide a method of forming a stent, comprising forming a plurality of patterned segments by coating at least part of a metal tube with a photoresist, patterning the photoresist using projection photolithography, and developing the photoresist with a developer; combining the plurality of patterned segments in a nested arrangement; and fusing at least a first one of the plurality of patterned segments to at least a second one of the plurality of patterned segments to form the stent. The pattern may be advantageously formed on the inner and/or outer diameter or surface of any of the patterned segments (e.g., on the inner diameter of an inner patterned segment, on the inner diameter of an outer patterned segment, on the outer diameter of an inner patterned segment, on the outer diameter of an outer patterned segment, or any combination thereof). Alternatively, the invention also provides a method of forming a nested stent, comprising forming a plurality of stent segments, combining the plurality of stent segments in a nested arrangement, and fusing at least a first one of the stent segments to at least a second one of the stent segments to form the nested stent. The segments may have an outer diameter of in a range of about 0.5 mm to 50 mm and a wall thickness in range of about 10-150 μm (e.g., 50-125 μm, 75-100 μm, or any value or range of values therein).

### BRIEF DESCRIPTION OF THE DRAWINGS

FIG. 1 shows an exemplary embodiment of a projection photolithography apparatus according to the present invention.

FIG. 2 is a diagram demonstrating how the bulk of the radiation from the projection photolithography apparatus can be focused on the inner diameter of a medical device.

FIG. 3 depicts an exemplary arc shaped profile of a stent strut that is etched from a material having a polygonal cross-section or a non-curved surface.

FIG. 4 shows a section of the wall of an exemplary stent according to the present invention, having grooves etched in the inner and outer surfaces of the strut nodes.

FIG. 5 is a diagram of an exemplary embodiment of a stent according to the present invention having grooves in both the struts and strut nodes, as well as an exemplary bellows-type groove arrangement on the strut nodes for increased flexibility and conformability.

FIG. 6 is an exemplary embodiment of a stent according to the present invention having grooves in the outer surface of the struts and grooves in both the outer and inner surfaces of the strut nodes. The image also shows a cross-section of a stent strut, demonstrating a curved inner surface of the struts and strut nodes.

FIG. 7 shows a portion of an exemplary stent according to the present invention, having rounded struts and strut nodes, which reduce blood flow interference, and a bellows-type groove arrangement on the strut nodes for that can increase flexibility and conformability.

FIG. 8 shows a portion of the wall of an exemplary stent according to the present invention, in which the struts have curved, wing-like cross-sections to reduce blood flow interference. The image also shows a strut node with a bellows-type groove arrangement in a flexed position, illustrating the flexibility created by the bellows-type groove arrangement.

FIG. 9 is a diagram of an exemplary open-ring stent design that includes a number of bridging connections, in accordance with the present invention.

FIG. 10 is a diagram of an exemplary stent according to the present invention having multiple open-cell segments fused together in a nested arrangement.

FIG. 11 is a diagram showing an air foil shape or design for exemplary struts in the presently disclosed medical devices.

FIG. 12 is a diagram of an exemplary stent according to the present invention having multiple segments in a layered and/or nested arrangement.

FIG. 13 is a diagram of an exemplary stent according to the present invention having multiple phase-shifted segments in a layered and/or nested arrangement.

### DETAILED DESCRIPTION

Reference will now be made in detail to various embodiments of the invention, examples of which are illustrated in the accompanying drawings. While the invention will be described in conjunction with certain embodiments, it will be understood that they are not intended to limit the invention to these embodiments. On the contrary, the invention is intended to cover alternatives, modifications and equivalents that may be included within the spirit and scope of the invention as defined by the appended claims. Furthermore, in the following disclosure, numerous specific details are given to provide a thorough understanding of the invention. However, it will be apparent to one skilled in the art that the present invention may be practiced without these specific details. In other instances, well-known methods, procedures, components, and circuits have not been described in detail, to avoid unnecessarily obscuring aspects of the present invention.

The present invention concerns a process and an apparatus to create customized geometries on all surfaces of a stent or other medical device. The projection photolithography apparatus described herein can be used to produce geometric patterns or surface topographies such as grooves, ridges, channels, holes, wells, curves, and other geometric patterns (e.g. parallelograms such as squares, rectangles and other trapezoids; triangles, pentagons, spirals, hexagons, etc.) on all surfaces of (e.g., both the ID and the OD) of stents or other cylindrical, tubular or curved-surface medical devices. These manufacturing innovations can result in stents that minimize endothelial surface disruption of blood flow, and that have improved flexibility and conformability.

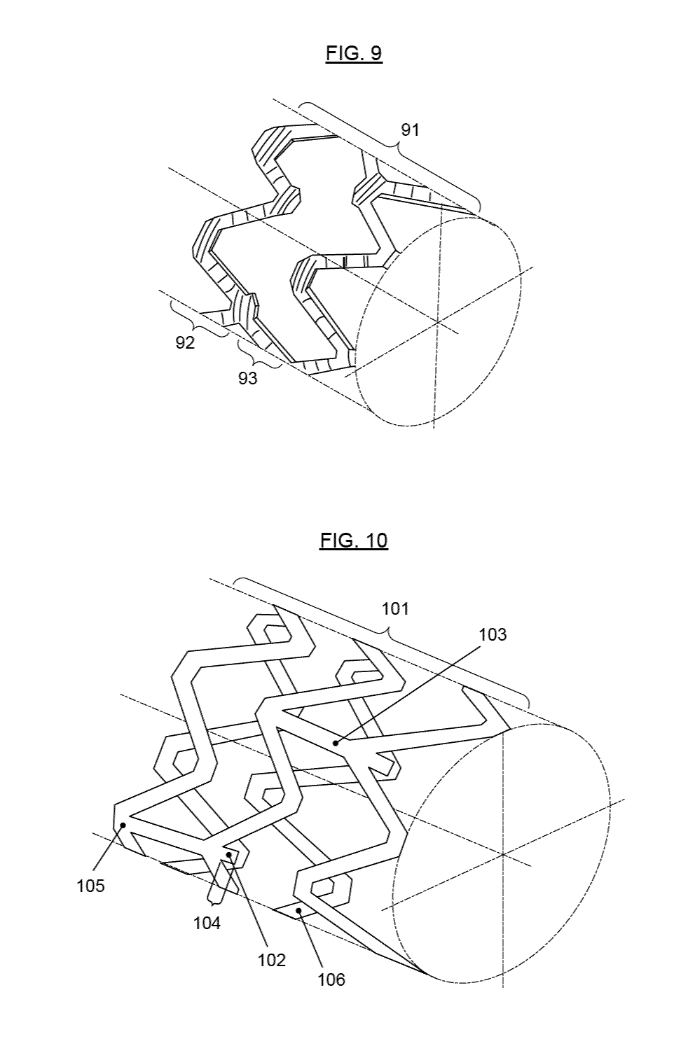

The present invention also presents a design and method for reducing stent strut diameter (thickness) by reducing strut dimensions with projection photolithography, and/or by incorporating two or more nested stents (e.g., stent layers, as shown in FIG. 9) in a concentric superstructure to form a scaffolded stent. The stent layers may be joined by one or more connection points that are formed using a bonding process, such as diffusion bonding, welding, etc. The stent cells (e.g., rings) in a given layer may be joined by one or more strut-like connectors (see, e.g., FIG. 10). Unlike most other commercial designs that utilize a single scaffold having many design elements, the present invention includes multiple, ultra-thin stent layers that are assembled into a larger superstructure that is implanted as a whole unit.

The present disclosure also includes the use of metallic substrates, namely high strength metallic alloys such as stainless steel, cobalt-alloys (e.g., cobalt-chromium alloys), tantalum, and NiTi alloys, although there are many other metals and alloys amenable to this process. Additionally, the process and apparatus can also be used over a wide variety of polymeric materials including biodegradable polymers such as PGA (e.g., poly[gluconic acid], poly[glucuronic acid], or a degradable copolymer thereof), PLA (e.g., poly[lactic acid] or a degradable copolymer thereof), etc. Each of these material substrates can be used to fabricate cylindrical tubes that provide an initial form, although the technology is not limited to tubular structures. Additional materials (e.g., plastics, composites, etc.) may be suitable for use with the present process and apparatus.

Using projection lithography to refine and form stent devices improves the optical resolution and critical dimensions of the devices, and avoids mask damage problems that result from the use of other photolithography techniques. The key difference between projection printing used in semiconductor industry and the present process and system for stent and medical device manufacturing is a very narrow area of the mask that can be imaged at the same time. Deviation of the cylindrical surface from a tangent plane at the center of a projected image should be smaller than the depth of focus within the projected image for effective patterning. In projection lithography, the pattern from the mask is projected by an imaging optical system onto the tube surface (inner or outer). A mask is positioned inside of the system, and can be manufactured at a different, larger scale (4:1, for example) relative to the desired pattern, which makes mask manufacturing simpler and reduces risk of contamination. Thus, while using diffraction limited imaging optics, extremely high resolution can be achieved. The resolution that is achievable in such system can be calculated by the following formula: CD=k₁·λ/NA (as described herein).

In another embodiment of the technology, it may be feasible and/or preferable to use an alternative to the photomask. For example, it may be possible to use an electro-optical component that includes a LiTaO₃ crystal. The crystal changes its optical properties in an applied electrical field. In a further alternative, a different version of a MEMS device (micro-electro-mechanical system) such as a grating light valve (GLV) could also be used in place of a photomask in the described system.

Based on the achievable micron-scale pattern resolution, projection lithography provides the smallest resolution features possible in stent manufacturing, enabling new designs and geometries that are specifically oriented to maximize vascular healing and/or minimize flow disruption over stent surfaces.

An Exemplary Projection Photolithography Apparatus

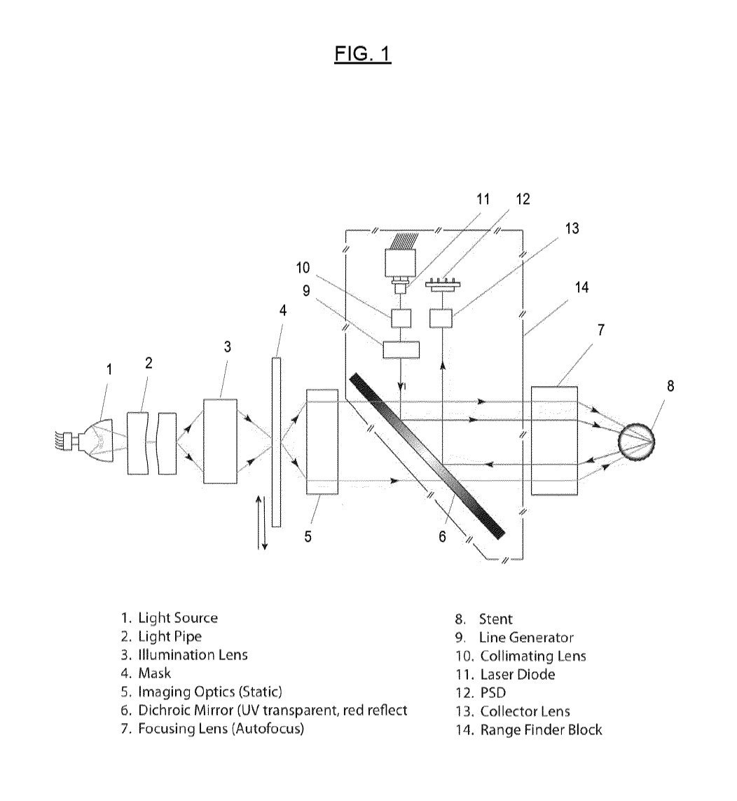

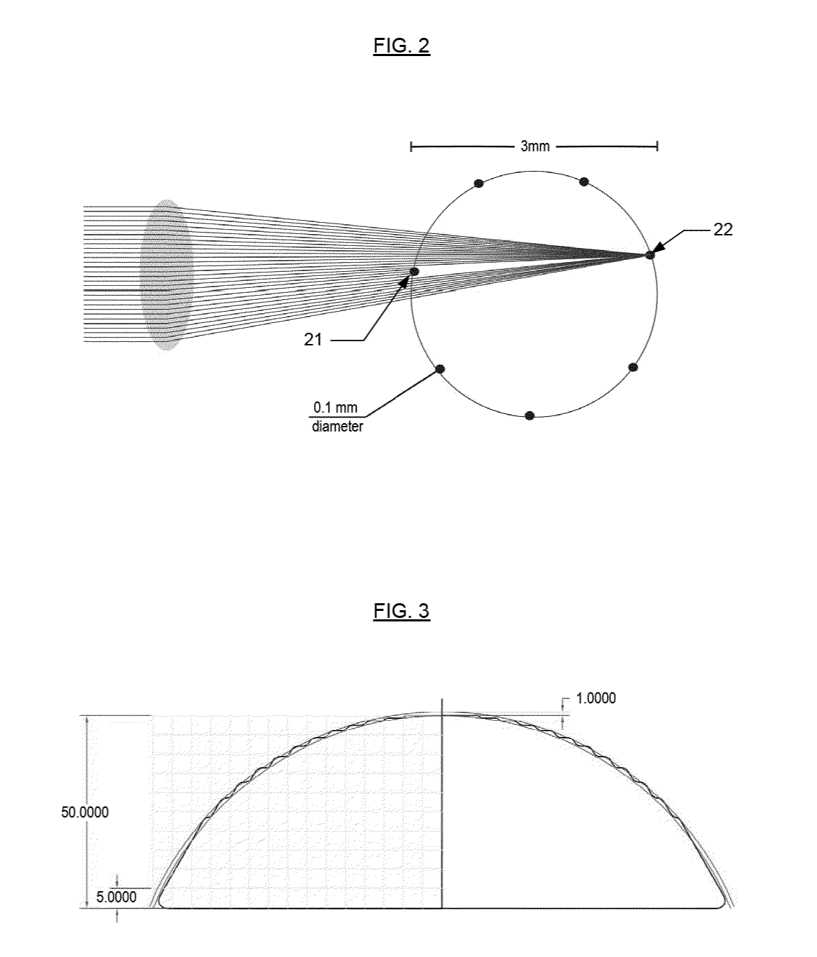

The present invention includes a projection photolithography apparatus for manufacturing and patterning stents and other medical devices. FIG. 1 provides a diagram of an exemplary projection photolithography apparatus. The exemplary projection photolithography apparatus includes an illuminator portion having a light source 1 that may be an excimer laser, a short arc mercury lamp, or other suitable light source. Light from the light source 1 is focused into a light homogenizer element 2, which can be a light pipe, but may also comprise any of a number of optical elements that can be combined to perform the function of a light homogenizer and ensure uniformity of mask illumination. Homogeneous light passing from the light homogenizer element 2 is collected by an illumination lens 3 that passes light through a photomask 4. The photomask 4 may contain a flattened (developed) pattern that can be projected onto the ID surface (or portion thereof) and/or the OD surface (or a portion thereof) of a medical device 8 (e.g., a stent).

The mask pattern being imaged generally has a rectangular shape, narrow in the vertical direction (limited by depth of focus) and a relatively long axial direction, although other shapes may be suitable (e.g., oval or irregular shapes, etc.). The mask pattern can be imaged onto the curved surface of the stent to be patterned by a synchronous motion of the mask in the vertical direction while rotating the stent around its central axis. During this process, the image is literally wrapped around the stent.

The imaging lens comprises two main components, a collimating lens 5 and a focusing lens 7. The focusing lens 7 is part of an autofocus system, and it is positioned on an ultra-precision stage (not shown) to permit continual movement and keep the waist or focal point of the imaging light on the surface of the stent 8 within the depth of focus. The same goal of keeping the waist of the imaging light (i.e., the focal point or focal depth) on the desired surface of the stent or medical device 8 can also be achieved if the lens 7 is kept in a static position and the stent 8 is placed on the ultra-precision mechanical stage. The distance between the imaging lens 7 and the surface of the medical device 8 should be controlled very accurately. Accordingly, projection lithography systems for imaging small and accurate features preferably have an auto focusing mechanism (as described below).

To track the position of the stent surface, a laser triangulation range finder 14 is built into the system. A dichroic mirror 6 is included in the system. In one embodiment, the dichroic mirror 6 is transparent for UV and deep blue light, but reflective for red light. The mirror 6 is placed in a collimated section of the beam, and a red laser diode 11 is used to project a sharp line through the focusing lens 7 onto the surface of the stent 8. This is accomplished by using a collimating lens 10 to reimage the beam, which is subsequently shaped into a thin line by a cylindrical lens 9. Once the light from the red laser diode 11 is reflected off the stent surface, the beam is reflected by the dichroic mirror 6 to a collector lens 13 on the Position Sensitive Device (PSD) 12. An alternative range finder design could be implemented based on the Focault (knife-edge) concept. In this case, the imaging light would be reflected back to the PSD with a knife-edge blocking part of the beam. Accordingly, the projected beam will move or project onto the PSD as soon as the system/stent moves out of focus.

After light passes through the mask 4, the static imaging optics are positioned such that the light passing from the illumination optics fills up the aperture of the static imaging lens 5. In one embodiment, UV light is used, and is able to pass through the dichroic mirror 6. The light the passes to the focusing lens 7 and is focused on the ID of the stent 8, where it patterns the photoresist layer on the ID of stent 8.

The above described embodiments of projection photolithography apparatus are not exclusive, and the present invention cover further iterations of such apparatus. For example, the present invention encompasses a projection photolithography apparatus that includes a high precision mechanical stage on which a mask pattern (e.g., mask 4 in FIG. 1) may be mounted, a high precision stage on which the focusing lens (e.g., focusing lens 7 in FIG. 1) may be mounted, and/or a high precision mechanical stage on which the medical device (e.g., stent 8 in FIG. 1) is mounted.

An Exemplary Manufacturing Process

In exemplary embodiments of a manufacturing process of a medical device (e.g., a stent) includes coating a base material (e.g., a metal or polymer tubing) with a photoresist, using an exemplary photoresist apparatus (as described above) to irradiate and pattern the photoresist; and developing the photoresist after the is patterned. The patterned photoresist can then be used as a mask in a subsequent etching process to remove one or more portions of the base material exposed by the patterned photoresist. This basic process encompasses several different embodiments of the inventive method for manufacturing a medical device.

In one embodiment, a metal tube is first coated with a photoresist. This step includes a pre-cleaning of the tube, applying a photoresist material to the tube, and then baking the applied photoresist to improve adhesion. This pre-exposure bake may be conducted at 90° C. to 120° C. (e.g., 100° C. to 110° C., or any value or range of values therein) for 1 to 10 mins. (e.g., 1 to 2 mins., or any value or range of values therein). The photoresist comprises a radiation (e.g., UV light) sensitive compound and can be classified as a positive or negative photoresist, depending on how it responds to radiation exposure. For a positive photoresist, the exposed regions become more soluble in a developing solution, and are thus more easily removed during development process. The net result is that images formed in positive photoresist are the same as those on the mask. Positive photoresist generally includes three components: one or more photosensitive compounds, a base resin, and one or more organic solvents. Prior to light exposure, the photoresist compound is insoluble in the developer solution. During and/or after exposure, the photosensitive compound absorbs radiation (e.g., UV light) in the exposed pattern areas, changes its chemical structure (e.g., by photoreaction or photochemically induced modification), and becomes soluble in the developer solution. After development, material underlying the exposed areas in the photoresist pattern is removed during a subsequent etching process.

For a negative photoresist, the exposed regions become less soluble in the developing solution, and the patterns formed in the negative resist are the reverse or complement of the mask patterns. Negative photoresists are polymers having one or more photoreactive groups, or that are combined with one or more photosensitive compounds (e.g., a photoinitiator or sensitizer). During exposure, the photosensitive compound(s) absorbs the optical energy (e.g., light), which initiates a photochemically driven polymer cross-linking reaction. The reaction causes cross-linking of the polymer molecules in the photoresist layer. The cross-linked polymer has higher molecular weight and becomes insoluble in the developer solution. After development, the unexposed areas of the photoresist layer (i.e., those areas not exposed to electromagnetic radiation) are removed.

Subsequently, the pattern of a photoresist pattern mask (e.g., mask 4 in FIG. 1) is transferred by an exemplary photolithography apparatus to the metal tubing. Specifically, a light source 1, which can be a UV radiation source (e.g., an excimer laser, a UV lamp, etc.) having wavelength(s) in a range of 200 to 440 nm (e.g., 350-440 nm, or any value or range of values therein), passes light through the light pipe to homogenize the light rays, and then through the illumination lens 3 to mask 4. The mask has a predetermined pattern that defines grooves (or other geometric features) and/or a strut structure (e.g., a surface topography) on the metal tubing and/or a medical device (e.g., a stent) to (i) promote reductions in blood flow interference, inflammation, and restenosis; (ii) promote favorable cell migration and adhesion; and/or (iii) generally improve the performance of the stent after it is deployed in the vessel.

As the light passes through mask 4, it is filtered by the mask 4 to provide patterned light to the ID or OD surfaces of the metal tubing. The filtered radiation then passes through the imaging optics 5, through the dichroic mirror 6 and the focusing lens 7, which focuses the patterned light onto the ID or OD surface of the metal or polymer tubing to illuminate the photoresist and transfer the mask pattern to the photoresist. The tubing is aligned with respect to the mask in the optical lithographic system as described herein, and the resist is then exposed to radiation (e.g., UV light).

After the photoresist is exposed to radiation and patterned, the metal tubing is flooded, immersed in, or washed with photoresist developer solution. If positive photoresist is used, the exposed resist is dissolved in the developer (if negative photoresist is used, unexposed areas are removed). The stent is then rinsed and dried. After development, a post-baking process may be conducted. The post-baking process can be conducted at a temperature of 100° C. to 175° C. (e.g., 120° C. to 140 for ° C., or any value or range of values therein, depending on the type of photoresist used) for a period of 1 to 30 minutes (e.g., 2 to 5 mins., or any value or range of values therein, depending on the type of photoresist used). The predetermined pattern at this point has been transferred to the photoresist to form a photoresist pattern, such that the portions of the metal tubing that are intended to be removed and/or patterned are exposed by the developed photoresist pattern.

The predetermined pattern is then transferred to the surface of the tubing by etching the exposed portions of the tubing, using the photoresist pattern as a mask. Etching is a process of removing the surface layer(s) of the metal tubing that are exposed through openings in the photoresist pattern. Etching processes fall into two main categories: wet and dry. Alternatively, these surface modifications can be created using a femtosecond laser, another method for inscribing micron scale features.

Wet etching is typically used for medical devices with feature sizes ≧3 μm. Below this size, the precision and control required for medical device (e.g., stent) manufacturing generally requires dry etching techniques. The mechanism of wet chemical etching generally involves three steps: (a) the etching reactants are transported by diffusion to the reactive surface (e.g., the surfaces of the metal tubing exposed by photoresist pattern); (b) a chemical reaction occurs at the surface; and (c) the products of the reaction between the surface of the metal tubing and the etching reactants are removed by diffusion. Chemical etching can be performed by immersing the metal tubing into an etchant solution or by spraying the etchant solution on the metal tubing. Spray etching typically has a much higher etching rate and better etching uniformity, and has generally replaced immersion etching for many applications.

Dry etching is a generic term that refers to etching techniques in which gases are the primary etching agents and/or media. Medical devices such as stents can be etched with greater precision using dry etch processes. There are generally three dry etching techniques: plasma, ion beam milling, and reactive ion etch (RIE). Plasma etching is conducted in a plasma etcher comprising a chamber, vacuum system, gas supply and a power supply. In a typical plasma etching process, metal tubings are loaded into the chamber and then the pressure inside the chamber is reduced (e.g., by a vacuum system). After a vacuum is established, the chamber is then back-filled with one or more etchant and/or carrier gas(es). A power supply creates a radio frequency (RF) field through electrodes in the chamber. The RF field energizes the gas mixture to a plasma state. In the energized plasma state, reactive species in the plasma attack the surfaces of the metal tubing that are exposed by the photoresist pattern, converting the exposed material into volatile components that are removed from the system (e.g., by the vacuum system). Etch rates are typically much lower than in a wet etching process, on the order of 1 μm/min. However, plasma etching does produce more anisotropic (e.g., directionally biased) etching profiles (e.g., structures having substantially vertical walls), and more precision.

Ion beam dry etching and/or sputter etching are physical etching processes, in which the stent surface is bombarded by ionized gas (e.g., of an inert gas such as argon). Thus, these processes do not use a reactive gas, unlike reactive plasma etching. Material is removed primarily by momentum transfer, rather than by chemical reaction. These processes are very anisotropic (e.g., having a high directional bias), but has poor selectivity (e.g., the photoresist may be removed at relatively high rate).

Reactive ion etching (RIE) systems combine elements of plasma etching and ion beam etching. The RIE system generally has a good selectivity (e.g., for removing exposed surfaces of underlying material relative to removing photoresist) and is a system of choice when very high resolution etching is required (e.g., where the medical device has a features of a size of <3 μm).

The manufacturing steps described above can be repeated as many times as necessary and/or desired in order to apply multiple patterns to an individual metal tubing. For instance, when manufacturing a stent with a design that includes complicated three-dimensional strut shapes (e.g., a curved surface), a multistep subtractive etching method may be required (e.g., iterative photoresist application, development, and etching steps).

The above-described process steps can also be combined with coating steps (e.g., depositing material on a stent in a predetermined or desired location by well-known deposition methods [e.g., CVD, PECVD, LPCVD, ALD, etc.]) when certain structures on or in the stent are being built up (e.g., layer by layer). Materials that are biocompatible and that interact favorably with vessel walls may be deposited on a stent or other medical device during the manufacturing process. For example, metals such as highly flexible metal alloys (e.g., cobalt-alloys, cobalt-chromium alloys, stainless steel, tantalum, nickel-titanium alloys, etc.), and polymers such as PGA (e.g., poly[gluconic acid], poly[glucuronic acid], or a degradable copolymer thereof) or PLA (e.g., poly[lactic acid] or a degradable copolymer thereof) may be deposited on the stent or medical device to add texture, topography, and/or materials that promote cell migration and adhesion.

Thus, the present invention also encompasses embodiments that include a number of etching and deposition steps. For instance, an embodiment of the present invention may include sequentially using a first photoresist mask having a first predetermined pattern, formed by projection photolithography, as an etch mask to remove material from a tubing and form surface modifications (e.g., grooves therein), and then forming and using as second photoresist mask having a second predetermined pattern, formed by projection photolithography, as a deposition mask for depositing material on the etched tubing. Further embodiments may include one or more etching steps and/or one or more deposition steps that may or may not use a photoresist mask patterned by projection photolithography.

In a further aspect, the present invention relates to cell patterning of medical devices using projection photolithography to encourage vascular healing by influencing cell migration. This process refers to the micro-patterning of two-dimensional (e.g., surface layers) or three-dimensional structures on the inner diameter and outer diameter of the medical device (e.g., a stent). Three dimensional structures, including, but not limited to, grooves, lines, projections, holes, tunnels, channels or other surface irregularities, may be formed on the ID and OD of a stent. The grooves or other three-dimensional structures may have a width in a range of 1 to 25 μm (e.g., 10 to 15 μm, or any value or range of values therein), and a depth of 1 to 15 μm (e.g., 3 to 5 μm, or any value or range of values therein). The grooves or other 3-D structures may accelerate cell migration and/or influence the directionality of growth in endothelial and smooth muscle cells. Also, cell adhesion is encouraged by irregularities in surface morphology (e.g., irregularities of a particular size, and for specific materials, perhaps of a particular physical and/or morphological orientation). For example, grooves oriented substantially perpendicular to the longitudinal axis of the stent may be ideal for encouraging endothelial cell growth in and/or along grooves formed on the ID of the stent. Additionally, the present invention enables micron and sub-micron sized topographies on the inner surface of medical devices such as stents (see, e.g., chapters 11, 17 in Greco, R. S., F. B. Prinz, and R. L. Smith, Nanoscale technology in biological systems. 2005, Boca Raton: CRC Press.), which may be particularly useful for three dimensional microencapsulation of islet cells (see page 437 of Nanoscale Technology in Biological Systems).

Also, 2-D structures, such as a surface layer (e.g., a polymer layer), can be added to the medical device, which may influence (promote or inhibit) cellular motility and cell adhesion. In various embodiments, these materials may be coated or otherwise formed in one or more layers on the inner and/or outer surfaces of the medical device. A the medical device may be coated entirely or selectively with layer of one or more biodegradable or non-biodegradable polymer materials. Examples of biodegradable polymers include polyglycolic acid/polylactic acid [PGLA], polycaprolactone [PCL], polyhydroxybutyrate valerate [PHBV], polyorthoesters [POE], and polyethyleneoxide/polybutylene terephthalate [PEO/PBTP]. Examples of nonbiodegradable polymers include polyurethane [PUR], silicone [SIL], and polyethylene terephthalate [PETP]. The medical device may additionally or alternatively be coated with an anticoagulant, antibiotic, endocrinological, or other physiologically active coating, such as heparin, coumadin, a taxane (e.g., taxol), an immunosuppressive antibiotic (e.g., rapamycin), a non-thrombogenic biological material (e.g., phosphorylcholine or bovine pericardium), etc. One or more of these materials may be coated on the medical device structure before and/or after three-dimensional patterning of the medical device.

The present invention is highly useful in creating micropores, channels, notches, depressions, and/or other surface modifications on curved surfaces, which is difficult or challenging to do using traditional photolithographic methods. These surface modifications (e.g., micropores and depressions) may be utilized for the delivery of therapeutic agents. Additionally, the interaction of a stent or other medical device as described herein with cells, thrombogenic agents, and other biological agents within a patient can be modified through coating or modifying the surface material of the stent or medical device.

Exemplary Medical Devices

The above-described photolithography apparatus and methods may be used to manufacture improved medical devices, particularly stents having improved flexibility and conformability and good radial strength. Additionally, these methods can produce stents with reduced strut thickness and superior surface features and contours that prevent interference with normal hemodynamic flow.

Significant improvements in stent flexibility, conformability and clinical performance can be driven at the “macro” scale by using strut configuration, strut thickness, choice of metals, ID and OD dimensions, and stent architecture (e.g., nested closed vs. open, coil vs. slotted vs. ring, etc.). However, significant enhancements in stent performance can result from the incorporation of “micro” scale features (e.g., grooves and patterns in the ID and OD of the struts) that change both the physical characteristics of the stent as well as the biological interface of the stent with the body. Additionally, the incorporation of hemodynamic and aerodynamic flow principles into strut design can result in significant enhancement of stent performance in terms of reducing flow disruptions that contribute directly to thrombosis, inflammation, and restenosis.

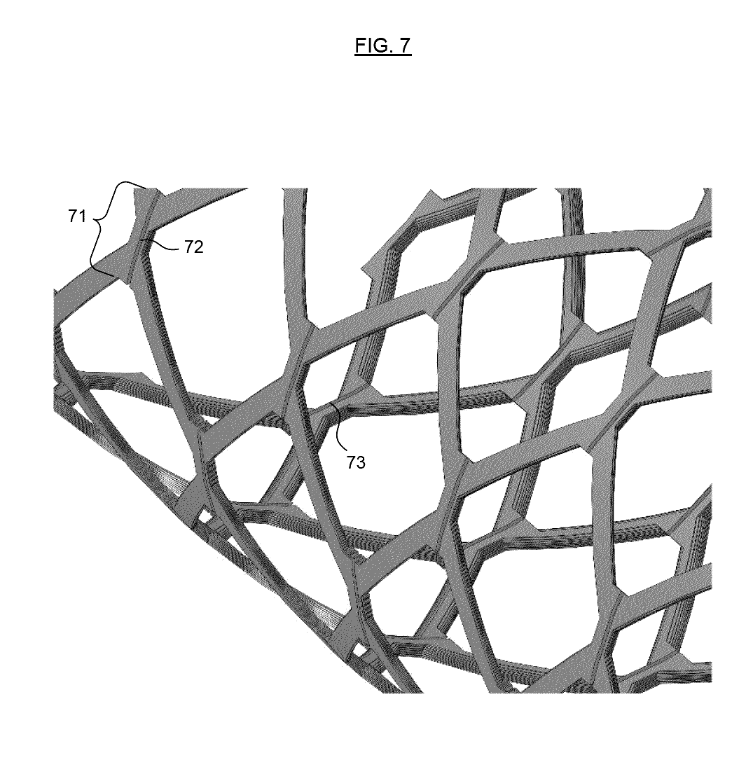

The “macro” features the stents disclosed herein include the thickness of the walls of the stents (e.g., the struts and intersections or nodes between the struts), which may be in a range of 10-150 μm (e.g., 50-125 μm, 75-100 μm, or any value or range of values therein). The length of the individual struts may be in a range of 0.1-3 mm (e.g., 1 mm, or any value or range of values therein). The struts may be formed in a diamond or hexagonal pattern to form the wall of the stent. As shown in FIGS. 6 and 7, the struts may form a mesh or fenestrated stent wall. However, the arrangement of the struts is not limited to the geometries shown in FIGS. 6 and 7. The struts may be arranged in other geometric or polygonal arrangements, and/or in arrangements having rounded portions.

The minimum outer diameter (OD) of the stents described herein may be about 0.5 mm when compressed (e.g., for insertion) and 1.0 mm when expanded (e.g., in the vessel). Typical stents may have a maximum expanded OD of up to about 4.5 mm (e.g., for coronary vessels), about 7-8 mm (e.g., for peripheral vessels), or about 35-38 mm (e.g., for aortic vessels), but a stent for treatment of an aortic aneurysm may have an OD much larger than 35 mm (e.g., up to 5 cm). The OD's of contracted stents may be from about 25% to about 50% of the expanded OD. The stents (the superstructure comprising multiple stent layers and/or segments) may have a total length in a range of 2 to 100 mm (e.g., 5-50 mm or any value or range of values therein).

Embodiments of the present invention include stent designs that comprise highly flexible metal alloys, such as cobalt-alloys, cobalt-chromium alloys, stainless steel, tantalum, nitinol, and other metals that allow strut thickness to significantly decrease without significant loss of radial strength. Alternatively, a wide variety of polymeric materials may be used, including biodegradable polymers such as PGA (e.g., poly[gluconic acid], poly[glucuronic acid], or a degradable copolymer thereof), PLA (e.g., poly[lactic acid] or a degradable copolymer thereof), etc. These metals and polymeric materials are believed to have long-term biocompatibility when embedded in arterial walls as stents.

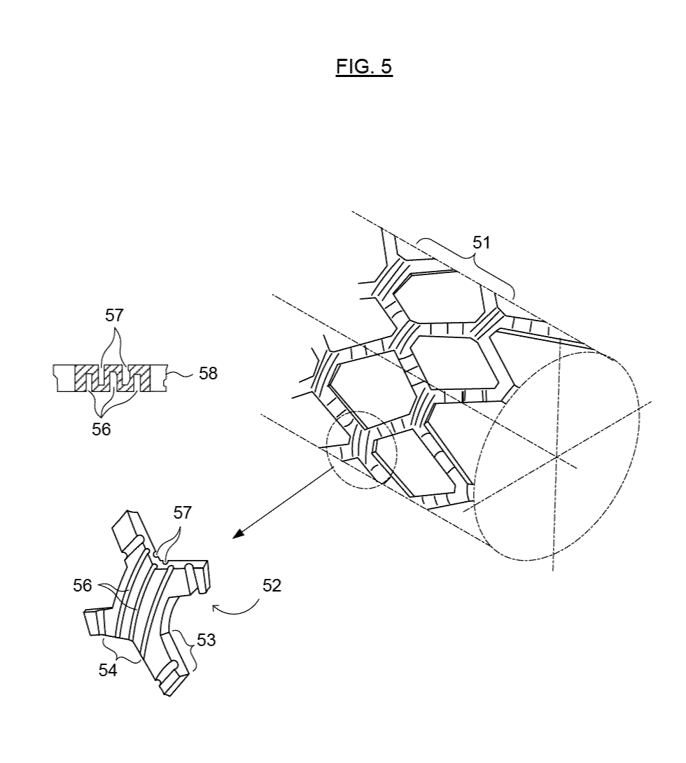

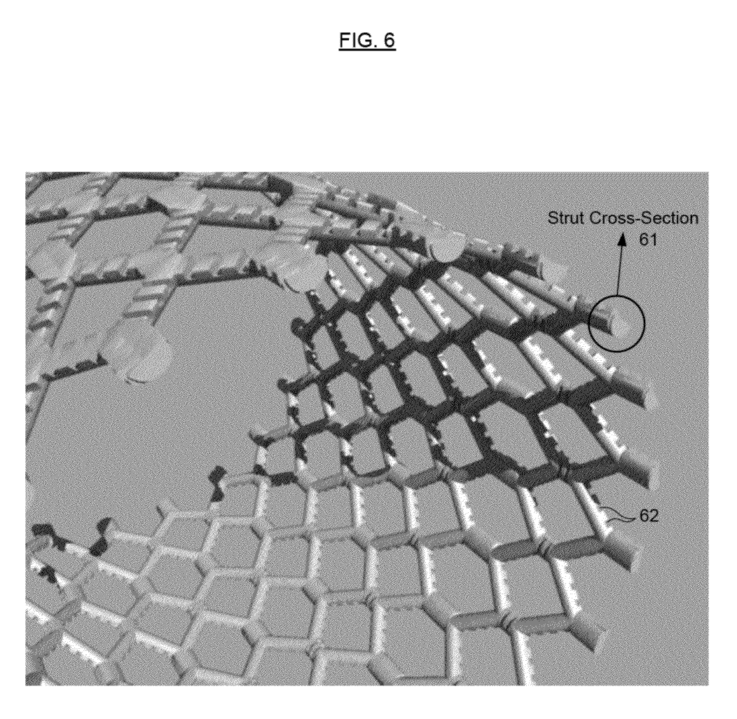

The stents disclosed herein include “closed cell” designs (see, e.g., FIG. 5), utilizing a number of connection points between adjacent stent struts to control the size of the stent cell area following deployment (e.g., in a blood vessel or other biological lumen or duct). Closed cell designs have a number of advantages including better retention of plaque and other vessel wall structures behind a closed meshwork. Also, closed cell designs provide more uniform drug delivery in stents designed for drug elution. FIG. 5 provides an illustration of a closed cell design having multiple, interconnected polygonal cells 51 that repeat along the circumference and length of the stent.

Alternatively, the stents encompassed by the present invention may have “open cell” designs (see, e.g., FIG. 9). The open cell provides more flexibility, but also suffer some drawbacks, such as “fish scaling,” in which large open-cell areas allow protrusion of the stent into the vessel wall, while also permitting prolapse of plaque and other materials into the lumen of the vessel. FIG. 9 provides an illustration of a stent segment or layer having opened cell design having multiple, zigzagging cells 91 (e.g., which may include one or more rings 92) having multiple struts that run along the circumference of the stent, which are interconnected by intermittent connecting struts 93.

An alternative, and innovative, stent architecture comprises multiple cylindrical components (e.g., stent layers) bonded together in a scaffolded arrangement (see, e.g., FIG. 10). By incorporating two or more nested, concentric stent layers (see, e.g., stent layers 105 and 106), connected by one or more bonding points 102, the strut diameter of the stent can be reduced without sacrificing the radial strength of the stent. The nested stent layers (e.g., stent layers 105 and 106) may be bound together at intersections 102 by diffusion bonding, welding, etc. FIG. 9 shows a stent cell 91, which may be combined with additional cells (e.g., in a repeating fashion) and incorporated into a superstructure 101 incorporating multiple stent layers. FIG. 10 shows the fused superstructure of a strut formed from multiple stent layers 105 and 106.

Each individual stent layer may have a length about equal to the length of the stent superstructure 101 (e.g., 2-100 mm, 5-50 mm or any value or range of values therein), and may have an open cell design. In an alternative embodiment, an individual stent layer may comprise a number of segments (e.g., segment 91 in FIG. 9), where each segment has a length that is less than the length of the stent superstructure 101 (e.g., 1 to 20 mm, or any value or range of values therein), and the segments of adjacent stent layers are arranged in a staggered pattern along the length of the stent, providing added flexibility. Each stent layer may have multiple (2-100, or any value or range of values therein) circumferential segments having multiple strut cells (2-100 or any value or range of values therein), spaced at a substantially constant distance from each other (e.g., 50-500 μm, or any value or range of values therein), where each of the segments has struts arranged in a zigzag pattern (see, e.g., FIGS. 9 and 10). This arrangement allows for the creation of a closed-cell lattice pattern when the stent layers are connected in the complete, layered stent superstructure 101 (see, e.g., FIG. 10). However, the stent layers are not limited to such a design (e.g., the stent layers may include sinusoidal or other strut cell patterns), and the strut cells may be spaced from one another at varying distances.

The stent layers also include connectors 103 for connecting strut cells within a stent layer and extensions 104 for forming connecting points 102 between adjacent stent layers (e.g., layers 105 and 106) to form the stent superstructure 101. The connectors 103 and extensions 104 may run longitudinally along the length of the stent superstructure 101. However, the connectors 103 and extensions 104 may be alternatively arranged at any angle relative to the longitudinal axis of the stent superstructure 101. Extensions 104 allow for a point at which adjacent stent layers may be fused at connecting points 102. As seen in FIG. 10, there are two concentric stent layers 105 and 106 pictured. Connectors 103 join adjacent cells in the same stent layer 105. Extension 104, when welded or otherwise connected to the adjacent stent layer 106, joins the adjacent stent layers 105 and 106 at connecting point 102, providing strength and/or stability to the overall stent superstructure 101, and allowing maximum stent expansion and flexibility while retaining a closed-cell architecture in the superstructure.

An advantage of the nested stent design is the fact that an open-cell design pattern (e.g., as shown in FIG. 9) can be used for each of the individual stent layers (e.g., stent layers 105 and 106), which provides improved (e.g., maximum) flexibility with or without the microfabrication patterns (e.g., grooves or other surface modifications). Ultra-thin struts can impart greatly improved flexibility to the structure by allowing movement between adjacent and layered stent struts. There may be one or more connection points 102 between adjacent stent cells of different stent layers (e.g., 1, 2, 3, or more). The flexibility of the struts may be minimally limited by connector points 102 at the proximal and distal ends of the individual stent layers, and points in between the ends, where the stent layers are bonded to form the superstructure 101.

The superstructure 101 comprising the concentric, open-cell stent layers (e.g., stent layers 105 and 106) will impart an overall closed-cell arrangement, meaning that there will be a substantially closed network of struts to constrain plaque and other materials from entering gaps/holes in the open cell design of the individual stent layers (see, e.g., FIG. 10). Two or more (e.g., 2, 3, 4, 5, or more) stent layers may be bonded in a nested superstructure to form the stent, such that the stent is 1, 2, 3, 4, or more layers thick along the stent. In other words, there may be as many as 3 or more stent layers overlapping at certain points of the stent, or substantially throughout the length of the stent. FIG. 10 provides an example where the stent superstructure 101 includes two layers of nested stent layers 105 and 106. In embodiments where there are more than two stent layers, the inner stent layer(s) are preferably bonded at one or more connection points per strut cell to each adjacent stent layer. Alternatively, where there are more than two stent layers, the inner stent layer(s) may be bonded intermittently (e.g., at least one connection point at every other strut cell) to each of adjacent stent layer. The above-described design elements accomplish the flexibility of an open-cell design with the retention of a closed-cell design. Should additional longitudinal flexibility be required, grooves, notches, channels, or other structures may be inscribed along the inner diameter (ID) and outer diameter (OD) of the stent, particularly on the nodal connections between adjacent stent cells or along the lengths of individual stent struts, as described herein. Those skilled in the art will appreciate that the above design is a combination of closed and open cell architectures that optimize flexibility and conformability.

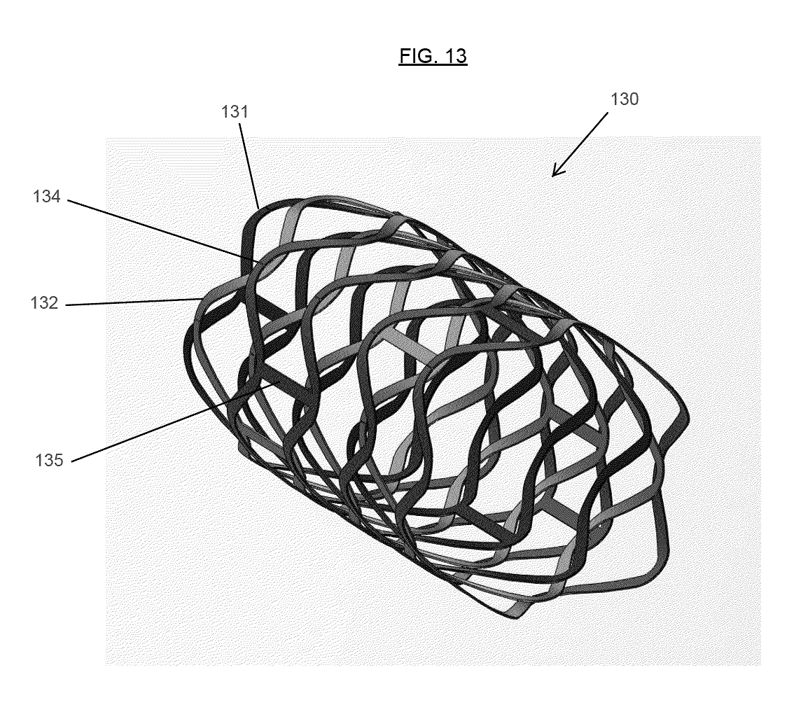

A further embodiment of an exemplary stent design 120 according to the present invention having multiple segments in a layered and/or nested arrangement is shown in FIG. 12. The green inner layer 122 is nested in the red outer layer 124. Each cell or ring in each of the inner and outer layers 122 and 124 is connected to an adjacent cell or ring by a stent 125. Although only one stent is shown connecting adjacent cells or rings, more than one stent can be used. Stents connecting adjacent cells or rings in a given layer 122 or 124 can be offset from each other by a predetermined amount (e.g., distance, angle and/or phase). As shown in FIG. 12, this amount may be about 150°, but any other amount can be used. In one example, the offset amount may depend on the number of stents (e.g., 360°/n±Δ, where n is the number of stents between adjacent cells or rings, and Δ is a predetermined phase offset less than 360°/n). Similarly, the stents in overlapping cells in adjacent layers may be offset by a predetermined amount (e.g., distance, angle and/or phase) in the same or similar manner, or in a complementary manner (e.g., if the offset between stents in adjacent cells or rings is 360°/n+Δ, the offset between stents in overlapping cells in adjacent layers may be 360°/n−Δ, where Δ≦360°/2n).

A still further embodiment of an exemplary stent design 130 according to the present invention having multiple phase-shifted segments in a layered and/or nested arrangement is shown in FIG. 13. The blue inner layer 131 is nested in the green center layer 132, which is in turn nested in the red outer layer 134. Each cell or ring in each of the inner, central and outer layers 131, 132 and 134 is connected to an adjacent cell or ring by a stent 135. Stents connecting adjacent cells or rings in a given layer 131, 132 or 134 are offset from each other by a predetermined amount, as are stents in overlapping cells of adjacent layers (e.g., 131 and 132, or 132 and 134). Part of each cell in the inner layer 131, the center layer 132, and the outer layer 134 (e.g., the lower half) is offset from the remainder of the cell (e.g., in the upper half of the layer as shown in FIG. 13). The offset may be a predetermined phase or distance, and may provide the stent with additional flexibility, conformability and/or radial strength. Although the design 130 of FIG. 13 shows a phase offset in each cell of each of the three layers 131, 132 and 134, the phase offset can be in less than all cells in a layer and/or in less than all of the layers.

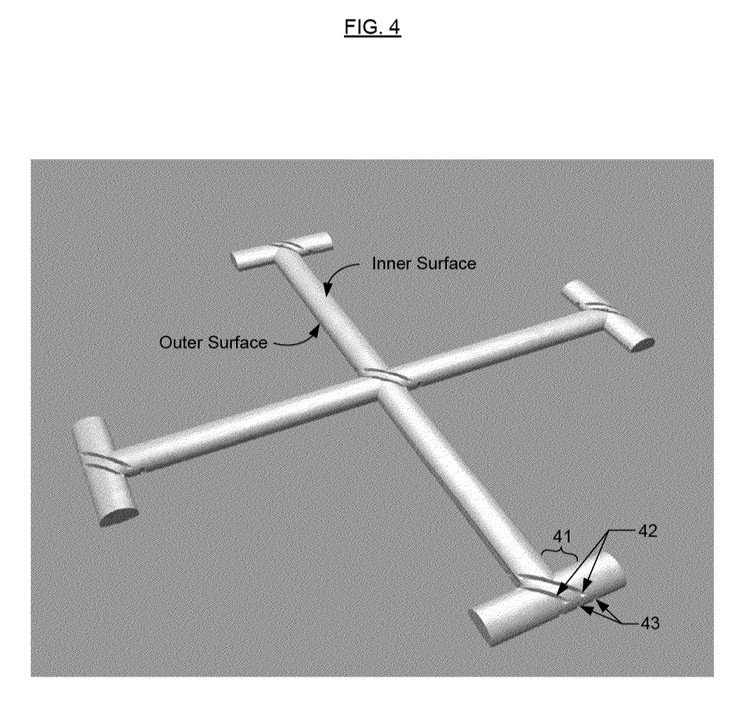

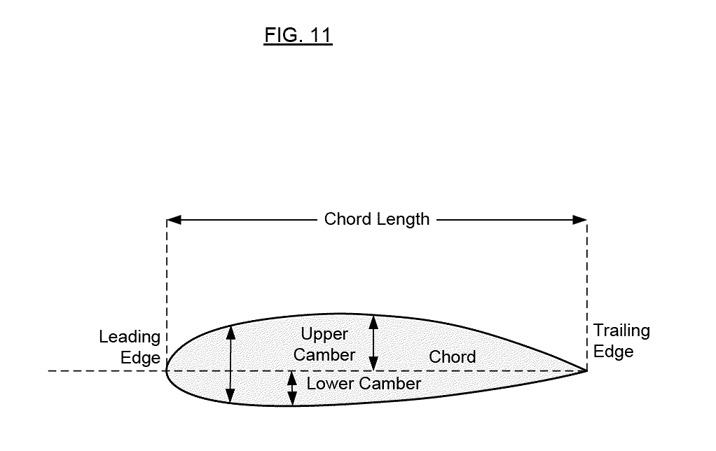

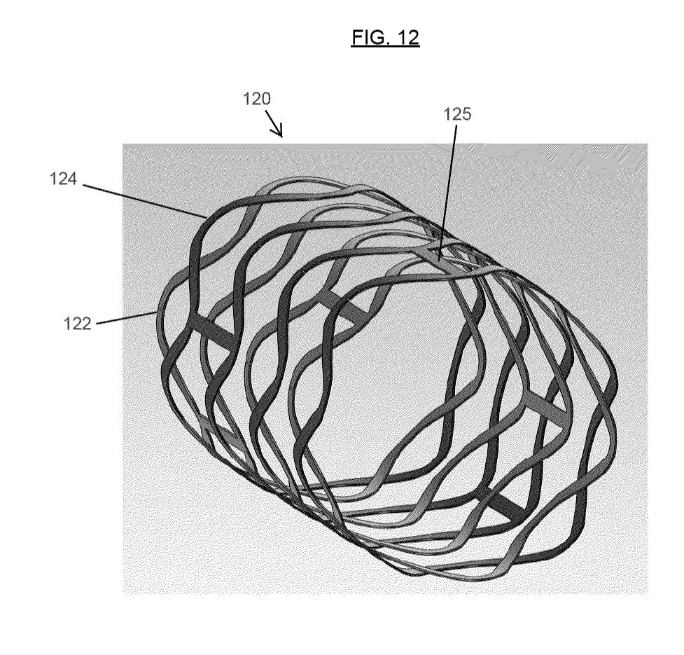

The embodiments of the present invention may also include an additional “macro” feature, the incorporation of curved surfaces on the ID and OD of the stent. In particular, featuring the inside diameter of stent struts with curved surfaces that approximate an airfoil, as shown in FIG. 11, or other curves that will result in less disruption of normal blood flow over endothelial surfaces around the stent struts. This flow disruption has been shown to be related to the formation of blood clots (thrombosis) and cellular proliferation (restenosis) that closes off vascular structures over time. Incorporating more hemodynamic/aerodynamic designs that blend aerodynamic and fluid dynamics principles should result in a significant diminution or elimination of these flow disruptions around stent struts, facilitating earlier healing and incorporation of stent struts into the vascular wall. These hemodynamic designs can involve modification of the strut geometry and may include one or more modifications to circumference, contour, diameter, and taper of the strut. These curved surfaces are represented in FIGS. 4 and 6-8.

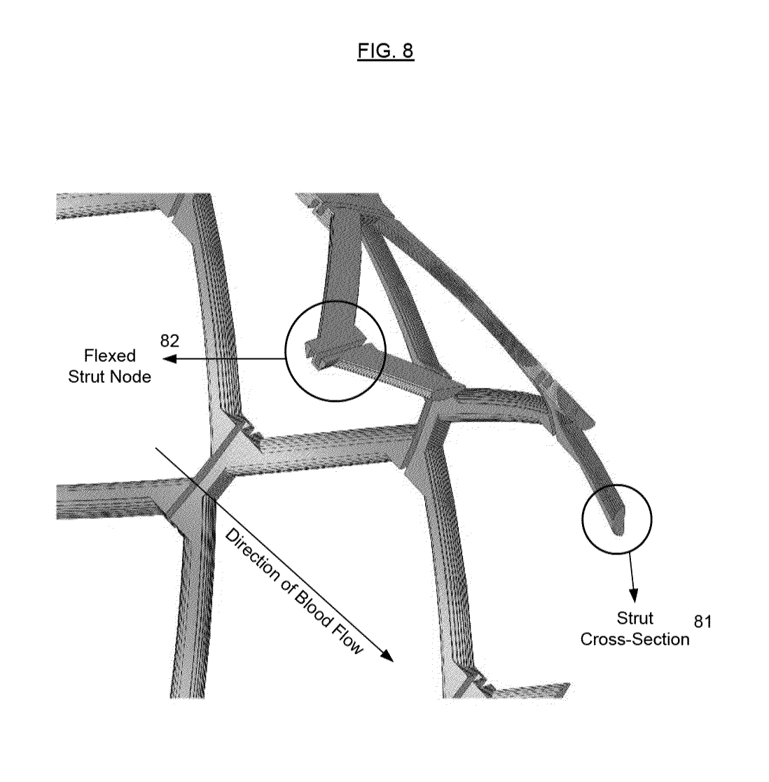

A curved surface for both the endoluminal and abluminal stent strut contour may minimize pressure on the endothelial cell surface while minimizing flow disturbances on the luminal surface. In this invention, the strut design is tailored such that the orientation of the stent strut in relation to the axis of blood flow can be normalized; the leading edge of the curved or arced surface (e.g., like the leading edge of an air foil) can be engineered to point as directly as possible into the blood flow to take fullest advantage of the hemodynamic surface properties of each stent strut. For example, FIG. 8 provides a close view of stent in order to show the curved cross section 81, which approximates an air foil shape, creating a smooth, arced surface over which blood flows. This design reduces or minimizes disruption of luminal blood flow. Also, as seen in the FIG. 6, the cross sectional area 61 of each strut is curved or arc-shaped on the ID, with significant variability in chord length, upper and lower camber and conformation of the leading edge. The chord length can be related to the overall thickness of the stent strut, although trailing edge and leading edge features may be added. The ID and OD of the stent nodes can be grooved and/or notched (see, e.g., ID grooves 42 and OD grooves 43 on the strut nodes 41 in FIG. 4) in order to maximize longitudinal flexibility and the OD of the stent struts can also have stent grooves (see, e.g., OD stent grooves 62 in FIG. 6) to optimize conformability of the stent struts during expansion of the stent, either through balloon expansion or self-expanding materials. In the example shown in FIG. 7, there is only one OD groove 72 on the OD of the stent nodes 71, and only one ID groove 73 on the stent nodes 71. The number of grooves may be as little as one or as many as four (or more) on both the ID and OD of the stent intersections or nodes. For example, FIG. 4 shows three OD grooves 43 and two ID grooves 42 on strut node 41.

One “micro” feature that provides superior flexibility to the stents of the present invention is the presence of grooves, channels, notches, depressions, and/or other surface modifications in the stent surface. The grooves or other surface modifications can alter mechanical deformability of the stent or stent struts, enhancing flexibility along an axial dimension. The surface modifications can create discrete points of improved flexibility and conformability along the length of a stent, and can be inscribed on both the inner diameter (ID) and outer diameter (OD) of the stent using projection photolithography, as described above. The photoresist mask may be formed in a predetermined pattern that includes grooves, channels, notches, depressions, and/or other surface modifications in the ID and/or OD stent surface. For instance, the predetermined pattern may include exposed regions of stent struts that define lateral grooves to be formed in a subsequent etching step.

One purpose of these grooves is to incorporate particular points of flexion (mechanical bellows) that permit significantly greater movement and flexion in the longitudinal direction while maintaining radial strength and resistance to fatigue. These flexion points may occur at the intersections (nodes) of stent struts (see, e.g., ID grooves 42 and OD grooves 43 on strut nodes 41 in FIG. 4), as well as along the OD and ID of the struts themselves. Surface modifications on the OD of the stent at points along the stent struts improve conformability of the stent by increasing deformability of the strut during stent installation and expansion, thus enabling struts to fit to vessel contours (see, e.g., strut grooves 62 in FIG. 6). FIG. 8 provides a depiction of flexion at a strut node 82 of an exemplary stent, where the mechanical bellows created by grooves formed in both the ID and the OD of the node provide improved flexion.

The grooves are inscribed grooves that may run perpendicularly and/or parallel to the longitudinal axis of the stent (see, e.g., FIGS. 5 and 6). The grooves may have various cross-sectional shapes (e.g., rectangular, U-shaped, V-shaped, hemispherical, elliptical, etc., any of which can be beveled or rounded at the edge if so desired), specific widths, lengths, and/or depths in order to optimize the longitudinal flexibility at the nodal sites. Specifically, these grooves or channels can be approximately 1 to 100 μm in width (e.g., 5 to 25 μm, or any value or range of values therein; in one embodiment, about 10 μm).

The grooves may be formed on the ID, OD, or both, depending on whether the grooves are formed along a strut or at a strut node (nodal grooves). For example, FIG. 5 shows an enlarged section 51 having strut grooves 55 on the OD of struts 53, and OD nodal grooves 56 and ID nodal grooves 57 on strut nodes 54. Grooves along the struts may contribute both to longitudinal flexibility and conformability by facilitating deformation of the stent struts in the radial curvature, without weakening the radial strength of the stent (see, e.g., FIGS. 5 and 6). The strut grooves (e.g., strut grooves 55) permit the stent struts to conform more easily to convex geometries, enhance the overall conformability of the stent, and minimize gap formation between strut and the body structure (e.g., an artery). There may be two or more grooves along the length of a strut (e.g., at least 2, 3, 4 or more, optionally up to about 10, 20, 30, or more; or any value or range of values therein), depending on the length of the individual struts. The grooves or other surface modifications are placed primarily on the outside diameter of the stent because their number, physical dimensions and orientation, when placed on the ID of the stent, could result in disruption of blood flow. However, in some embodiments, the grooves or surface modifications may be located on both the ID and the OD of the struts.

The dimensions of the strut grooves (e.g., strut grooves 55) may be different than those of nodal grooves (e.g., nodal grooves 56 and 57, see FIGS. 5 and 6). Strut grooves may be wider than nodal grooves, ranging between 5-100 μm (e.g., 20-50 μm, or any value or range of values therein). The depths of the strut grooves may range from 5-75 μm (e.g., 10-50 μm, or any value or range of values therein), or alternatively, from 5 to 75% of the thickness of the strut (or any value or range of values therein). The orientation of these grooves may be directly perpendicular to the longitudinal axis of the stent or placed at some other angle relative to the longitudinal axis, depending on the desired direction of conformability. For instance, the grooves may be formed perpendicularly to the length of the strut in which they are formed (see, e.g., strut grooves 62 in FIG. 6). The angle of the strut grooves can be 20° to 90° (e.g., 70° to 90°, or any value or range of values therein), relative to the longitudinal axis of the stent, regardless of the geometric arrangement of the struts themselves, whether they be arranged in a diamond, hexagonal, or other pattern. There may be stents configured for particular anatomies that require either more or less conformability, and there is some optimal combination of groove angle, depth, width, number and placement that may facilitate particular anatomies or challenging routes of delivery.

As mentioned above, there may also be one or more strut nodes 41 inscribed with nodal grooves 42 and 43, as illustrated in FIG. 4. FIG. 4 shows the ID of strut junctions where two struts cross. In this example, there can be two or more nodal grooves 42 running perpendicular to the longitudinal axis of the stent on the ID (e.g., at least 2, 3, 4 or more, optionally up to about 10, 20, 30, or more; or any value or range of values therein) and three or more nodal grooves 43 running perpendicular to the longitudinal axis of the stent on the OD of each node (e.g., at least 3, 4, 5 or more, optionally up to about 10, 20, 30, or more; or any value or range of values therein). In one embodiment, the number of perpendicular grooves on the OD of a node is one more than the number of perpendicular grooves on the ID of the node, and the grooves on the OD are staggered with the grooves on the ID, thereby forming a bellows-like arrangement. For example, FIG. 5 shows a cross-section 58 of a strut node 54 having three nodal grooves 56 on the OD of the strut node 54 and two nodal grooves 57 on the ID of the strut node 54. The nodal grooves may have a depth that is 10 to 75% of the strut node thickness (e.g., 40% to 70%, or any value or range of values therein). Preferably, the grooves have a depth of at least 55% of the strut node thickness. For instance, in the case of a stent strut having a thickness of 100 μm, these grooves would preferably be at least 55 μm in depth on both the OD and ID of the nodal junction of the stent, as shown in FIGS. 4 and 5.

Alternatively or additionally, there may be one or more nodes inscribed with longitudinal grooves, running parallel to the longitudinal axis of the stent. The nodal intersections can each be notched with three or more of the longitudinal grooves (e.g., 3 to 30, or any value or range of values therein) on the OD, with each groove being 1 to 50 μm in diameter (e.g., 5 to 25 μm, 10-15 μm, or any value or range of values therein), and having a depth greater than at least 55% of the strut thickness. The longitudinal grooves may contribute to conformability by facilitating deformation of the stent struts in the radial curvature. In further embodiments, some nodes may have perpendicular grooves, and other nodes may have longitudinal grooves (e.g., alternating nodes along a cross-section of the stent perpendicular to the longitudinal axis of the stent). In addition, some nodes may not have any grooves, while other nodes have either longitudinal and/or perpendicular grooves.

The struts and nodes on the stents of the present invention may also have holes or grooves, known as “wells”, that open onto the exterior surface and are considered suitable for containing one or more therapeutic agents. The wells are described as variable in depth, opening onto the OD of the stent, or passing through to the interior (ID) of the stent and containing therapeutic material.

A further “micro” feature that is incorporated into embodiments of the present invention are surface patterns on the ID and OD surfaces to encourage vascular healing. Specifically, it is known in the literature that endothelial and smooth muscle cells have an accelerated rate and directionality of migration when encountering grooves and other surface irregularities. Using projection photolithography, numerous patterns including, but not limited to grooves, lines, projections, holes, tunnels, channels or other surface irregularities may be formed on the ID and OD of a stent. This process refers to the micro-patterning of 2-D (e.g. surface layers) or 3-D structures (e.g., small grooves, lines, holes, etc.) on the surface of the medical device (e.g., stent struts) that influence cell migration based on an individual cell's response to a microenvironment and the surface of the medical device. In the case of 3-D structures, the grooves or lines may have a width in a range of 1 to 25 μm (e.g., 10 to 15 μm, or any value or range of values therein), and a depth of 1 to 15 μm (e.g., 3 to 5 μm, or any value or range of values therein). For example, an orientation substantially perpendicular to the longitudinal axis of the stent may be ideal for encouraging endothelial cell growth in and/or along grooves formed on the ID of the stent. The present invention enables micron and sub-micron sized topographies on the inner surface of medical devices such as stents (e.g., stent struts; see, e.g., chapters 11 and 17 in “Nanoscale Technology in Biological Systems” cited herein) which may be particularly useful for three dimensional microencapsulation of islet cells (see page 437 of “Nanoscale Technology in Biological Systems”). A similar reference to the use of surface topography to influence cell growth and endothelial cell coverage is contained in U.S. Pa. No. 6,190,404, which refers to the use of at least one groove disposed substantially parallel to the longitudinal axis of the stent.

In the case of 2-D structures, such as a surface layer, cellular motility and cell adhesion can be influenced by certain materials that encourage or discourage cell adhesion. In various embodiments, these materials may be coated or otherwise formed in one or more layers on the inner and/or outer surfaces of the medical device. In one case, cell adhesion is encouraged by irregularities in surface morphology (e.g., irregularities of a particular size, and for specific materials, perhaps of a particular physical and/or morphological orientation). A medical device may be coated entirely or selectively with layer of one or more biodegradable or non-biodegradable polymer materials. Examples of biodegradable polymers include polyglycolic acid/polylactic acid [PGLA], polycaprolactone [PCL], polyhydroxybutyrate valerate [PHBV], polyorthoesters [POE], and polyethyleneoxide/polybutylene terephthalate [PEO/PBTP]. Examples of nonbiodegradable polymers include polyurethane [PUR], silicone [SIL], and polyethylene terephthalate [PETP]. The medical device may additionally or alternatively be coated with an anticoagulant, antibiotic, endocrinological, or other physiologically active coating, such as heparin, coumadin, a taxane (e.g., taxol), an immunosuppressive antibiotic (e.g., rapamycin), a non-thrombogenic biological material (e.g., phosphorylcholine or bovine pericardium), etc. One or more of these materials may be coated on the medical device structure before and/or after three-dimensional patterning of the medical device.

The grooves and surface modifications described above can be formed through the projection photolithography methods described above. For instance, the photoresist mask may define grooves or channels that run horizontally and/or longitudinally to the longitudinal axis of the stent. Furthermore, multiple horizontal grooves or channels may be formed on both the ID and OD of the strut intersections, such that a “bellows-type” flexible joint 58 is created (see, e.g., FIG. 5). The bellows-type joint 58 allows increased flexibility and motion in the longitudinal direction while maintaining radial strength. Some embodiments of the present invention include bellows-type joints 58 as shown in FIG. 5, where the grooves at the strut intersections can be formed in an alternating pattern with a nodal groove 57 on the ID following a nodal groove 56 on the OD along the length of the strut node 54.

### CONCLUSION/SUMMARY

The present invention is directed to stents and other medical devices that can have specific geometric configurations (curves, arcs, contours, tapers) thereon, methods of making such stents and other medical devices, and apparatuses for making such stents and other medical devices. Applications include, but are not limited to vascular stents used in treating diseases including arterial and venous patency in atherosclerotic vascular disease, pulmonary arterial stenoses, coarctation, and pulmonary and systemic venous obstruction, among other applications. The application of projection photolithography allows for the production of customized geometry/contours on all surfaces of stent struts, achieving a manufacturing solution for producing stents designed specifically to minimize endothelial surface disruption of blood flow. More specifically, the above-described methods may be used to manufacture medical devices, in particularly stents, having reduced strut thickness and superior surface features and contours that prevent interference with normal hemodynamic flow and improved flexibility and conformability without necessarily sacrificing radial strength.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the claims appended hereto and their equivalents.

What is claimed is:

1. A method of forming patterned surface on a medical device, comprising:
a) coating at least part of the medical device with a photoresist;
b) transferring a pattern to the photoresist using projection photolithography; and
c) developing the photoresist with a developer to form a patterned photoresist on the medical device.

2. The method of claim 1, further comprising selectively removing a material of the medical device or part thereof exposed by the patterned photoresist, or selectively adding a new material to a surface of the medical device or part thereof exposed by the patterned photoresist.

3. The method of claim 2, wherein the photoresist is applied to an inner surface and an outer surface of the medical device, and the pattern is transferred to portions of the photoresist on the inner surface and the outer surface of the medical device.

4. The method of claim 1, wherein transferring the pattern to the photoresist comprises aligning the medical device with respect to a mask having a representation of the pattern thereon, and passing radiation through the mask, thereby exposing the resist to radiation in the pattern of the mask.

5. The method of claim 1, wherein the medical device or part thereof has a tubular or cylindrical shape.

6. The method of claim 5, wherein the medical device is a stent.

7. The method of claim 1, wherein the medical device or part thereof includes a number of fenestrations.

8. An apparatus for making a medical device, comprising:
a) a radiation source providing radiation;
b) a range finder configured to enable locating a surface of the medical device;
c) a focusing lens for focusing the radiation beam onto the medical device; and
d) a first mechanical stage configured to move the medical device rotationally and/or along at least one of two orthogonal axes, a first one of the orthogonal axes being parallel with an optical axis of the apparatus, the first mechanical stage having sufficient precision to enable focusing the radiation on either inner surface of the medical device under a first set of imaging conditions and on an outer surface of the medical device under a second set of imaging conditions.

9. The apparatus of claim 8, further comprising a light pipe or a light homogenizer configured to receive the radiation from the radiation source and homogenize the light.

10. The apparatus of claim 8, further comprising a second mechanical stage configured to move the mask, and/or a third mechanical stage configured to move the focusing lens.

11. A medical device, comprising:
a) one or more cylindrical bodies having a mesh-like wall including struts and strut nodes; and
b) a first pattern of surface features on inner surfaces of the struts and/or strut nodes.

12. The medical device of claim 11, wherein an outer diameter of the medical device is in a range of from 0.5 mm to 50 mm, and a thickness of the wall is in a range of from 10 to 150 μm.

13. The medical device of claim 11, further comprising a second pattern of surface features on outer surfaces of the struts and/or strut nodes.

14. The medical device of claim 11, wherein a cross-section of the struts has a curved or airfoil shape.

15. The medical device of claim 13, wherein the surface features of the first pattern and the second pattern include one or more inner grooves, ridges, channels, holes, and/or wells.

16. The medical device of claim 15, wherein the inner grooves, ridges, or channels have dimensions and an orientation facilitating cell migration, cell adhesion, and/or drug delivery.

17. A medical device, comprising:
a) a first cylindrical body having a first mesh-like wall including struts and strut nodes; and
b) a second cylindrical body having a second mesh-like wall including struts and strut nodes;
wherein the first and second cylindrical bodies are bonded together in a concentric nested arrangement.

18. The medical device of claim 17, wherein each of the first and second cylindrical bodies has a length that is less than a length of the medical device.

19. The medical device of claim 17, wherein each of the first and second cylindrical bodies are arranged in a staggered arrangement along the length of the medical device.

20. The medical device of claim 17, wherein at least one of the first and second cylindrical bodies includes at least one phase-shifted cell.

21. The medical device of claim 17, wherein at least one of the first and second cylindrical bodies includes a plurality of stents that are phase-shifted with respect to each other or with respect to at least one stent in an adjacent cylindrical body.

22. The medical device of claim 17, wherein at least one of the first and second cylindrical bodies has a first pattern of surface features on inner surfaces of the struts and/or strut nodes.
